(19) United States
(12) Patent Application Publication
(10) Pub. No.: US 2022/0204426 A1
(43) Pub. Date: Jun. 30, 2022

(54) DOWNSTREAM PRODUCTION PROCESS FOR HIGH PURITY BUTADIENE

(71) Applicant: SABIC Global Technologies B.V., Bergen op Zoom (NL)

(72) Inventors: Muhammad H. HAIDER, Riyadh (SA); Abdulaziz AL-ZAHRANI, Riyadh (SA); Te CHANG, Sugar Land, TX (US)

(73) Assignee: SABIC Global Technologies B.V., Bergen op Zoom (NL)

(21) Appl. No.: 17/610,647

(22) PCT Filed: May 19, 2020
(86) PCT No.: PCT/IB2020/054746

§ 371 Date: Nov. 11, 2021

Related U.S. Application Data
(60) Provisional application No. 62/855,723, filed on May 31, 2019.

(51) Int. Cl. C07C 7/08; B01J 21/08; B01J 23/62; B01J 23/78; C07C 5/333; C07C 7/00; B01D 3/40; B01D 3/14

(52) U.S. Cl. CPC C07C 7/08; B01J 21/08; B01J 23/62; B01J 23/78; C07C 5/3337; C07C 2523/78; B01D 3/40; B01D 3/143; C07C 2521/08; C07C 2523/62; C07C 7/005

(57) ABSTRACT

Systems and methods for producing butadiene are disclosed. In a reaction unit, n-butane is dehydrogenated in the presence of a double-dehydrogenation catalyst to produce a mixture that includes butadiene and unreacted n-butane. An extractive distillation unit that uses soybean oil as the solvent is utilized to extract at least some of the unreacted n-butane from the mixture.

### CROSS REFERENCE TO RELATED APPLICATIONS

This application claims the benefit of U.S. Provisional Patent Application No. 62/855,723 filed May 31, 2019, which is expressly incorporated by reference herein in its entirety.

### FIELD OF INVENTION

The present invention generally relates to systems and methods for producing butadiene. More specifically, the present invention relates to systems and methods for producing butadiene via a single-step double-dehydrogenation process and purifying the produced butadiene via a series of separation steps that include an extractive distillation step with soybean oil as the solvent.

### BACKGROUND OF THE INVENTION

Demand for butadiene (BD) is increasing due to its use as monomer for polymers, plastics, synthetic rubber or elastomers, noticeable of which are styrene butadiene rubber (SBR), polybutadiene rubber (PBR), polychloroprene (neoprene) and nitrile rubber (NR). Globally, most of the BD is produced by steam cracking of ethylene, which obtains BD as a by-product from naphtha feedstock. Another competing technology for BD production includes dehydrogenation of normal butenes feedstock, which can give reasonable yields. However, BD production from n-butane surpasses both of the previously listed processes in terms of economics, as n-butane is an economical feed source.

Conventionally, n-butane is used as the feedstock to produce butadiene via multiple dehydrogenation steps with different reaction conditions and/or catalyst. Overall, the multi-step approaches require high capital expenditure and high operational costs. Furthermore, the butadiene purification/separation process after the dehydrogenation steps includes multiple extractive distillation steps using highly toxic solvent(s) and multiple distillation steps to obtain highly purified butadiene. These purification and/or separation steps can result in high energy consumption and high environment impact for producing butadiene.

Overall, while systems and methods for producing butadiene using n-butane as the feedstock exist, the need for improvements in this field persists in light of at least the aforementioned drawbacks for conventional systems and methods.

### BRIEF SUMMARY OF THE INVENTION

A solution to at least some of the above-mentioned problems associated with the production process for butadiene has been discovered. The solution resides in a method of producing butadiene via a single-step dehydrogenation of n-butane. This can be beneficial for at least using a more economical feedstock than conventional methods that use butenes as a feed source. The single-step dehydrogenation further reduces the operational costs and the capital expenditure required for producing butadiene compared to conventional methods. Additionally, the produced mixture from the dehydrogenation step containing butadiene is further separated via a series of steps, including an extractive distillation step that uses soybean oil as the solvent. Because soybean oil is more environmentally friendly than the organic solvent used in the conventional methods, the present method can reduce the environmental impact of producing BD compared to the conventional methods. Furthermore, the disclosed methods are capable of improving the energy efficiency for separating/purifying butadiene from the effluent of the single-step dehydrogenation process, resulting in further reduced production costs for butadiene. Therefore, the method of the present invention provides a technical solution to at least some of the problems associated with the currently available methods for producing and purifying butadiene.

Embodiments of the invention include a method of separating butadiene. The method comprises providing a mixture comprising butadiene and unreacted n-butane. The method comprises contacting the mixture with soybean oil under conditions such that the n-butane dissolves in the soybean oil at a higher rate than the butadiene to form (1) a first stream comprising the soybean oil and at least some of the n-butane of the mixture and (2) a second stream comprising primarily butadiene.

Embodiments of the invention include a method of producing butadiene. The method comprises dehydrogenating n-butane to produce a mixture comprising butadiene and unreacted n-butane. The method comprises contacting the mixture with soybean oil under conditions such that the unreacted n-butane dissolves in the soybean oil at a higher rate than the butadiene to form (1) a first stream comprising the soybean oil and at least some of the unreacted n-butane of the mixture and (2) a second stream comprising primarily the butadiene. The conditions include a temperature of 10 to 50° C. The method comprises separating the second stream into a third stream comprising primarily butadiene, a fourth stream comprising C₁ to C₃ hydrocarbons, a fifth stream comprising primarily butane and butenes, and a sixth stream comprising hydrocarbons having a higher boiling point than butadiene.

Embodiments of the invention include a method of producing butadiene. The method comprises dehydrogenating n-butane to produce a mixture comprising butadiene and unreacted n-butane. The method comprises contacting the mixture with soybean oil under conditions such that the unreacted n-butane dissolves in the soybean oil at a higher rate than the butadiene to form (1) a first stream comprising the soybean oil and at least some of the unreacted n-butane of the mixture and (2) a second stream comprising primarily the butadiene. The conditions include a temperature of 10 to 50° C. The method comprises separating the second stream into a third stream comprising primarily butadiene, a fourth stream comprising C₁ to C₃ hydrocarbons, a fifth stream comprising primarily butane and butenes, and a sixth stream comprising hydrocarbons having a higher boiling point than butadiene. The method comprises separating the first stream to produce a seventh stream comprising primarily n-butane and an eighth stream comprising primarily soybean oil. The method comprises routing the eighth stream to a dehydrogenation reactor that carries out the dehydrogenation.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The term “double dehydrogenation”, as that term is used in the specification and/or claims, means a single dehydrogenation step that transforms two single bonds of a hydrocarbon molecule into two double bonds.

The term “Cₙ+ hydrocarbon” wherein n is a positive integer, e.g. 1, 2, 3, 4, or 5, as that term is used in the specification and/or claims, means any hydrocarbon having at least n number of carbon atom(s) per molecule.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a schematic diagram of a system for producing butadiene, according to embodiments of the invention; and

FIG. 2 shows a schematic flowchart of a method of producing butadiene, according to embodiments of the invention.

### DETAILED DESCRIPTION OF THE INVENTION

Currently, butadiene can be produced by dehydrogenation of butene or multi-step dehydrogenation of n-butane. The use of butene as the feedstock for producing butadiene has not been economically feasible due to the high cost of butene. Multi-step dehydrogenation of n-butane to produce butadiene generally requires high capital expenditure and high production costs. Additionally, the separation/purification process for purifying butadiene produced via the multi-step dehydrogenation process has high environmental impact and requires a large amount of energy. The present invention provides a solution to at least some of these problems. The solution is premised on a method of producing butadiene using n-butane as feedstock. The method includes a single-step dehydrogenation process to produce butadiene, thereby reducing the capital expenditure and production costs for producing butadiene compared to conventional methods. Moreover, the disclosed method uses soybean oil as the solvent in at least one of the extractive distillation steps, resulting in reduced pollution risks compared to conventional methods. These and other non-limiting aspects of the present invention are discussed in further detail in the following sections.

### A. System for Producing Butadiene

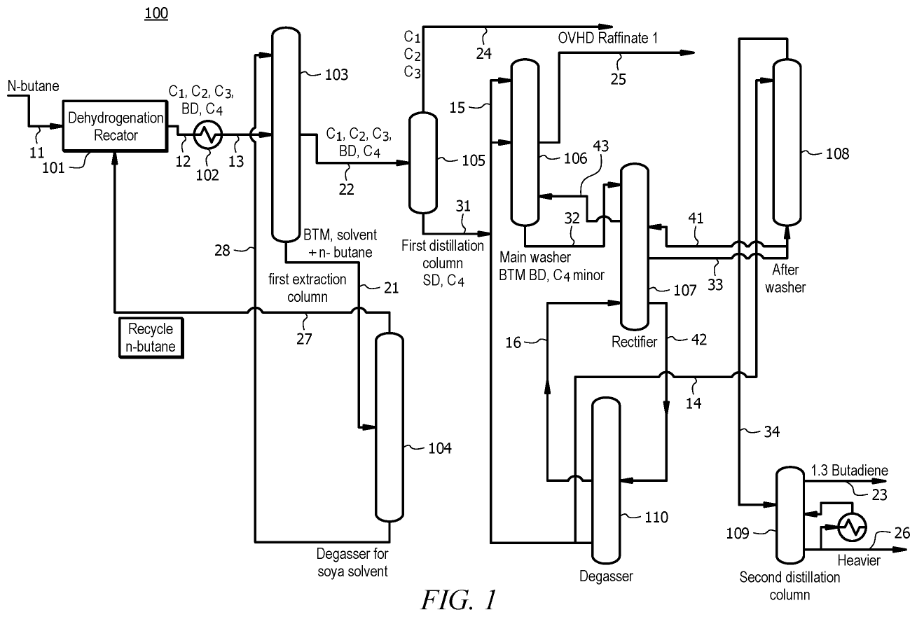

In embodiments of the invention, the system for producing butadiene can include a reaction unit for dehydrogenating n-butane to produce butadiene, at least two extractive distillation units, one or more distillation columns, and one or more degassers. With reference to FIG. 1, a schematic diagram is shown of system 100 for producing butadiene. According to embodiments of the invention, system 100 includes reaction unit 101 configured to receive feed stream 11 comprising n-butane and dehydrogenate n-butane to produce butadiene via a single-step dehydrogenation process.

In embodiments of the invention, reaction unit 101 includes one or more fixed bed reactors. Reaction unit 101 may contain one or more catalyst capable of catalyzing double-dehydrogenation of n-butane to form butadiene. The catalyst may include a Column 13 or Column 14 metal (of the Periodic Table) or oxide(s) thereof. The catalyst can be supported on an iron-stabilized alkaline earth metal-silica support. According to embodiments of the invention, non-limiting examples of catalytic metals (noble metals and Columns 13 and 14 of the Periodic Table) include rhodium (Rh), palladium (Pd), ruthenium (Ru), platinum (Pt), gold (Au), gallium (Ga), indium (In), germanium (Ge), antimony (Sb), and bismuth (Bi), oxides thereof, alloys thereof and mixtures thereof. Non-limiting examples of alkaline earth metals (Column 2 of the Periodic Table) include magnesium (Mg), calcium (Ca), strontium (Sr), barium (Ba), and combinations thereof.

The Fe-alkaline earth metal-silica support can include 0.5 to 3 wt. % iron and all ranges and values there between including 0.5, 1, 1.5, 2, 2.5, and 3 wt. %. The Fe-alkaline earth metal-silica support can include 20 to 40 wt. % alkaline earth metal and all ranges and values there between including ranges of 20 to 25 wt. %, 25 to 30 wt. %, 30 to 35 wt. %, and 35 to 40 wt. %. In embodiments of the invention, silicon and oxygen make up the balance in the Fe-alkaline earth metal-silica support. The catalyst in reaction unit 101 can include up to 20 wt. % of the total amount of total catalytic transition metal, from 0.001 wt. % to 20 wt. %, from 0.01 wt. % to 15 wt. %, or from 1 wt. % to 10 wt. % and all wt. % or at least, equal to, or between any two of 0.001 wt. %, 0.01 wt. %, 0.1 wt. %, 1 wt. %, 2 wt. %, 3 wt. %, 4 wt. %, 5 wt. %, 6 wt. %, 7 wt. %, 8 wt. %, 9 wt. %, 10 wt. %, 15 wt. %, and 50 wt. %, with the balance being support material.

In some embodiments, the catalyst in reaction unit 101 includes gallium and palladium. The molar ratio of Ga:Pd in Fe-alkaline metal-silica supported catalyst or physical mixture can be in a range of 0.01 to 0.5 and all ranges and values there between including ranges of 0.01 to 0.05, 0.05 to 0.1, 0.1 to 0.15, 0.15 to 0.2, 0.2 to 0.25, 0.25 to 0.3, 0.3 to 0.35, 0.35 to 0.4, 0.4 to 0.45, and 0.45 to 0.5. Overall, the active catalyst may have a composition of 3 to 20 wt. % palladium, 0.05 to 8 wt. % gallium, 40 to 80 wt. % silica, and 0.05 to 8 wt. % iron.

According to embodiments of the invention, an outlet of reaction unit 101 is in fluid communication with cooler 102 such that effluent stream 12 flows from reaction unit 101 to cooler 102. Effluent stream 12 may comprise butadiene, unreacted n-butane, butenes (including 1-butene, 2-butene and/or isobutene), C₁ to C₃ hydrocarbons, C₅+ hydrocarbons. Cooler 102 may be configured to cool effluent stream 12 to produce cooled effluent stream 13. In embodiments of the invention, cooler 102 may include heat exchangers. In embodiments of the invention, an outlet of cooler 102 is in fluid communication with an inlet of first extractive distillation unit 103 such that cooled effluent stream 13 flows from cooler 102 to first extractive distillation unit 103. First extractive distillation unit 103 is configured to process cooled effluent stream 13 via extractive distillation to produce first stream 21 comprising a first solvent and at least some of the unreacted n-butane and second stream 22 comprising butadiene, butenes (including 1-butene, 2-butene and isobutene), n-butane, C₁ to C₃ hydrocarbons, and C₅+ hydrocarbons. In embodiments of the invention, the first solvent includes soybean oil. First stream 21 may comprise more than 33 wt. % of the unreacted n-butane from cooled effluent stream 13.

In embodiments of the invention, a bottom outlet of first extractive distillation unit 103 is in fluid communication with an inlet of first degasser 104 such that first stream 21 flows from first extractive distillation unit 103 to first degasser 104. First degasser 104 may be configured to separate first stream 21 into seventh stream 27 comprising n-butane and eighth stream 28 comprising primarily the first solvent. According to embodiments of the invention, an outlet of first degasser 104 is in fluid communication with an inlet of reaction unit 101 such that seventh stream 27 flows from first degasser 104 to reaction unit 101. An outlet of first degasser 104 may be in fluid communication with an inlet of first extractive distillation unit 103 such that eighth stream 28 comprising primarily the first solvent flows from first degasser 104 to first extractive distillation unit 103.

According to embodiments of the invention, an outlet of first extractive distillation unit 103 is in fluid communication with first distillation unit 105 such that second stream 22, comprising butadiene, butenes (including 1-butene, 2-butene and isobutene), n-butane, C₁ to C₃ hydrocarbons, and C₅+ hydrocarbons, flows from first extractive distillation unit 103 to first distillation unit 105. First distillation unit 105 is configured to separate second stream 22, via distillation, to produce fourth stream 24 comprising primarily C₁ to C₃ hydrocarbons and first intermediate stream 31 comprising butadiene, n-butane, butenes, and C₅+ hydrocarbons. In embodiments of the invention, first distillation unit 105 may comprise one or more distillation columns, each of which has a theoretical plate number in a range of 60 to 80 and all ranges and values there between including ranges of 60 to 62, 62 to 64, 64 to 66, 66 to 68, 68 to 70, 70 to 72, 72 to 74, 74 to 76, 76 to 78, and 78 to 80.

In embodiments of the invention, a bottom outlet of first distillation unit 105 may be in fluid communication with second extractive distillation unit 106 such that first intermediate stream 31 flows from first distillation unit 105 to second extractive distillation unit 106. Second extractive distillation unit 106 is configured to separate first intermediate stream 31 via extractive distillation to produce (a) fifth stream 25 comprising primarily butane and butenes and (b) second intermediate stream 32 comprising butadiene, a second solvent, and less than 5 to 15 wt. % other C₄ hydrocarbons including butenes, and n-butane. In embodiments of the invention, second extractive distillation unit 106 uses a second solvent comprising N-Methyl-2-pyrrolidone, 5-11% water, or combinations thereof.

In embodiments of the invention, a bottom outlet of second extractive distillation unit 106 is in fluid communication with rectifier zone 107 such that second intermediate stream 32 flows from second extractive distillation unit 106 to rectifier zone 107. Rectifier zone 107 is configured to remove butenes from second intermediate stream 32 to form (1) third intermediate stream 33 comprising butadiene, other C₄ hydrocarbons, the second solvent, and C₅+ hydrocarbons, (2) first loop stream 42 comprising primarily C₄ hydrocarbons and the second solvent, collectively, and (3) second flow back stream 43 comprising non-butadiene C₄ hydrocarbons and the second solvent. Second flow back stream 43 may flow from rectifier zone 107 back to second extractive distillation unit 106. In embodiments of the invention, rectifier zone 107 is capable of removing substantially all of the butenes from second intermediate stream 32. In embodiments of the invention, rectifier zone 107 comprises packed beds of solid material capable of separating BD from other C₄ hydrocarbons.

According to embodiments of the invention, rectifier zone 107 is in fluid communication with after-washer zone 108 such that third intermediate stream 33 flows from rectifier zone 107 to after-washer zone 108. After-washer zone 108 may be configured to separate third intermediate stream 33 comprising butadiene, other C₄ hydrocarbons, the solvent, and C₅+ hydrocarbons to produce (1) fourth intermediate stream 34 comprising butadiene and C₅+ hydrocarbons and (2) first flow back stream 41 comprising primarily C₄ hydrocarbons and the second solvent, collectively. First flow back stream 41 may be flowed from after-washer zone 108 back to rectifier zone 107. In embodiments of the invention, after-washer zone 108 uses the second solvent as a solvent for separating third intermediate stream 33. According to embodiments of the invention, rectifier zone 107 and after-washer zone 108 may be two sections of a dividing wall column.

According to embodiments of the invention, an outlet of after-washer zone 108 is in fluid communication with second distillation unit 109 such that fourth intermediate stream 34 flows from after-washer zone 108 to second distillation unit 109. Second distillation unit 109 may be configured to separate fourth intermediate stream 34 to produce (1) third stream 23 comprising primarily butadiene and (2) sixth stream 26 comprising primarily C₅+ hydrocarbons. In embodiments of the invention, second distillation unit 109 may comprise one or more distillation columns, each of which has a theoretical plate number in a range of 70 to 80 and all ranges and values there between including 70, 71, 72, 73, 74, 75, 76, 77, 78, 79, and 80.

According to embodiments of the invention, an outlet of rectifier zone 107 is in fluid communication with second degasser 110 such that first loop stream 42 flows from rectifier zone 107 to second degasser 110. Second degasser 110 may be configured to separate first loop stream 42 to produce (a) recycle solvent stream 15 comprising the second solvent, (b) after-washer solvent stream 14, and (c) recycle C₄ stream 16 comprising C₄ hydrocarbons and the second solvent. In embodiments of the invention, an outlet of second degasser 110 may be in fluid communication with second extractive distillation unit 106 and/or after-washer zone 108 such that recycle solvent stream 15 and/or after-washer solvent stream 14 flow from second degasser 110 to second extractive distillation unit 106 and/or after-washer zone 108. In embodiments of the invention, an outlet of second degasser 110 may be in fluid communication with an inlet of rectifier zone 107 such that recycle C₄ stream 16 comprising C₄ hydrocarbons and the second solvent flows from second degasser 110 to rectifier zone 107. In embodiments of the invention, an outlet of second degasser 110 may be in fluid communication with a cooling column or heat exchanger such that recycle C₄ stream 16 flows to the cooling column and/or heat exchanger. The cooling column and/or heat exchanger are configured to cool recycle C₄ stream 16.

### B. Method of Producing Butadiene

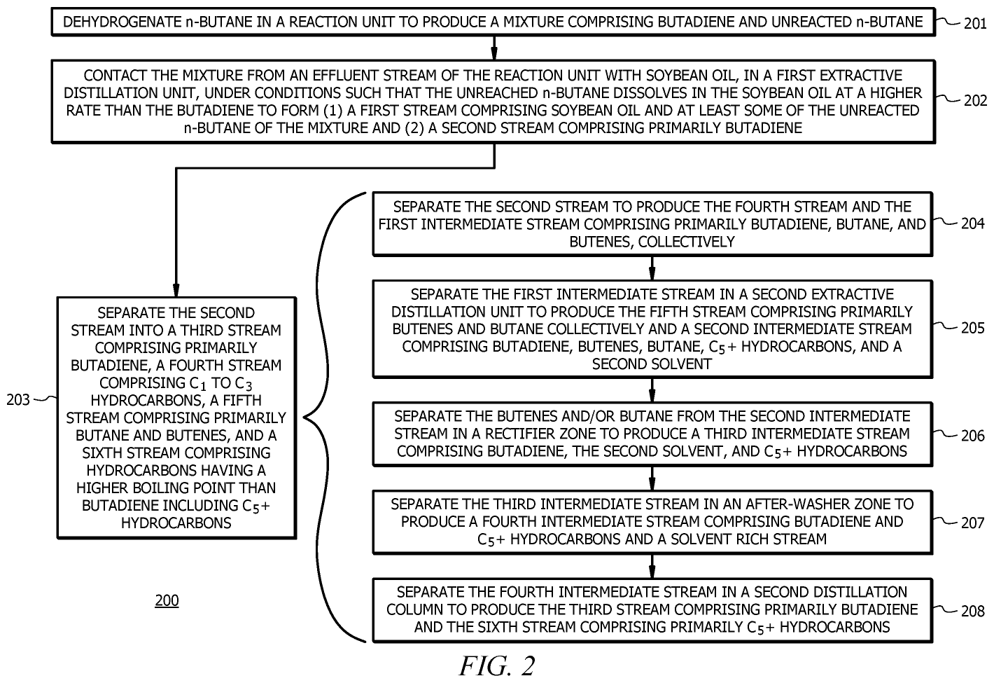

Methods of producing butadiene that includes double dehydrogenating n-butane to produce a mixture that contains butadiene and using soybean oil as a solvent in an extractive distillation unit for separating the butadiene have been discovered. Embodiments of the methods are capable of reducing the use of toxic solvent for extractive distillation and reducing the overall energy consumption for producing butadiene compared to conventional methods. As shown in FIG. 2, embodiments of the invention include method 200 for producing butadiene. Method 200 may be implemented by system 100, as shown in FIG. 1.

According to embodiments of the invention, as shown in block 201, method 200 includes dehydrogenating n-butane in reaction unit 101 to produce a mixture comprising butadiene and unreacted n-butane. In embodiments of the invention, the dehydrogenating at block 201 includes double-dehydrogenation of n-butane. In embodiments of the invention, the mixture produced at block 201 further comprises butenes including 1-butene, 2-butene, isobutene, or combinations thereof. The mixture produced at block 201 may further comprise C₁ to C₃ hydrocarbons and/or C₅+ hydrocarbons. The dehydrogenating at block 201 can be carried out in the presence of a catalyst in reaction unit 101, which includes a Column 13 or Column 14 metal or oxide thereof and a noble metal deposited on an iron alkaline earth metal-silicon oxide support.

According to embodiments of the invention, dehydrogenating at block 201 is carried out at a temperature of 450 to 600° C. and all ranges and values there between, including ranges of 450 to 455° C., 455 to 460° C., 460 to 465° C., 465 to 470° C., 470 to 475° C., 475 to 480° C., 480 to 485° C., 485 to 490° C., 490 to 495° C., 495 to 500° C., 500 to 505° C., 505 to 510° C., 510 to 515° C., 515 to 520° C., 520 to 525° C., 525 to 530° C., 530 to 535° C., 535 to 540° C., 540 to 545° C., 545 to 550° C., 550 to 555° C., 555 to 560° C., 560 to 565° C., 565 to 570° C., 570 to 575° C., 575 to 580° C., 580 to 585° C., 585 to 590° C., 590 to 595° C., and 595 to 600° C. The dehydrogenating at block 201 may be carried out at a pressure of 0.1 to 1 MPa and all ranges and values there between including ranges of 0.1 to 0.2 MPa, 0.2 to 0.3 MPa, 0.3 to 0.4 MPa, 0.4 to 0.5 MPa, 0.5 to 0.6 MPa, 0.6 to 0.7 MPa, 0.7 to 0.8 MPa, 0.8 to 0.9 MPa, and 0.9 to 1.0 MPa. The dehydrogenating at block 201 may be carried out at a weighted hourly space velocity in a range of 1000 to 3000 hr⁻¹ and all ranges and values there between including ranges of 1000 to 1100 hr⁻¹, 1100 to 1200 hr⁻¹, 1200 to 1300 hr⁻¹, 1300 to 1400 hr⁻¹, 1400 to 1500 hr⁻¹, 1500 to 1600 hr⁻¹, 1600 to 1700 hr⁻¹, 17000 to 1800 hr⁻¹, 1800 to 1900 hr⁻¹, 1900 to 2000 hr⁻¹, 2000 to 2100 hr⁻¹, 2100 to 2200 hr⁻¹, 2200 to 2300 hr⁻¹, 2300 to 2400 hr⁻¹, 2400 to 2500 hr⁻¹, 2500 to 2600 hr⁻¹, 2600 to 2700 hr⁻¹, 27000 to 2800 hr⁻¹, 2800 to 2900 hr⁻¹, and 2900 to 3000 hr⁻¹.

In embodiments of the invention, effluent stream 12 from reaction unit 101 containing the mixture may be cooled in cooler 102 to form cooled effluent stream 13. Cooled effluent stream 13 may be at a temperature in a range of 50 to 70° C. and all ranges and values there between including ranges of 50 to 52° C., 52 to 54° C., 54 to 56° C., 56 to 58° C., 58 to 60° C., 60 to 62° C., 62 to 64° C., 64 to 66° C., 66 to 68° C., and 68 to 70° C. According to embodiments of the invention, cooled effluent stream 13 may be flowed to first extractive distillation unit 103. In embodiments of the invention, as shown in block 202, method 200 comprises contacting the mixture of cooled effluent stream 13 with soybean oil, in first extractive distillation unit 103, under conditions such that the unreacted n-butane dissolves in the soybean oil at a higher rate than the butadiene to form (1) first stream 21 comprising soybean oil and at least some of the unreacted n-butane of the mixture and (2) second stream 22 comprising primarily butadiene. First stream 21 may comprise more than 30 to 60 wt. % n-butane from the mixture. Second stream 22 may comprise 60 to 80 wt. % butadiene and all ranges and values there between including ranges of 60 to 62 wt. %, 62 to 64 wt. %, 64 to 66 wt. %, 66 to 68 wt. %, 68 to 70 wt. %, 70 to 72 wt. %, 72 to 74 wt. %, 74 to 76 wt. %, 76 to 78 wt. %, and 78 to 80 wt. %. Second stream 22 may further comprise butenes, C₁ to C₃ hydrocarbons, C₅+ hydrocarbons, or combinations thereof.

According to embodiments of the invention, the conditions of contacting at block 202 include a temperature of 10 to 50° C. and all ranges and values there between including ranges of 10 to 12° C., 12 to 14° C., 14 to 16° C., 16 to 18° C., 18 to 20° C., 20 to 22° C., 22 to 24° C., 24 to 26° C., 26 to 28° C., 28 to 30° C., 30 to 32° C., 32 to 34° C., 34 to 16° C., 36 to 38° C., 38 to 40° C., 40 to 42° C., 42 to 44° C., 44 to 46° C., 46 to 48° C., and 48 to 50° C. In embodiments of the invention, the conditions of contacting at block 202 include a pressure in a range of 3 to 5 MPa and all ranges and values there between including ranges of 3 to 3.2 MPa, 3.2 to 3.4 MPa, 3.4 to 3.6 MPa, 3.6 to 3.8 MPa, 3.8 to 4.0 MPa, 4.0 to 4.2 MPa, 4.2 to 4.4 MPa, 4.4 to 4.6 MPa, 4.6 to 4.8 MPa, and 4.8 to 5.0 MPa. The conditions of contacting at block 202 may include a feed (cooled effluent stream 13) to solvent (soybean oil) flow rate ratio in a range of 0.10 to 0.20 and all ranges and values there between including 0.10 to 0.12, 0.12 to 0.14, 0.14 to 0.16, 0.16 to 0.18, and 0.18 to 0.20.

According to embodiments of the invention, as shown in block 203, method 200 comprises separating second stream 22 into third stream 23 comprising primarily butadiene, fourth stream 24 comprising C₁ to C₃ hydrocarbons, fifth stream 25 comprising primarily butane and butenes, and sixth stream 26 comprising hydrocarbons having a higher boiling point than butadiene including C₅+ hydrocarbons. In embodiments of the invention, as shown in block 204, separating at block 203 includes separating second stream 22 to produce fourth stream 24 comprising C₁ to C₃ hydrocarbons and first intermediate stream 31 comprising primarily butadiene, butane, and butenes, collectively. Separating at block 204 may be carried out in first distillation unit 105. First distillation unit 105 may be operated with a temperature of 75 to 85° C. and all ranges and values there between including 76° C., 77° C., 78° C., 79° C., 80° C., 81° C., 82° C., 83° C., and 84° C. First distillation unit 105 may be operated at an operating pressure of 3 to 5 bar and all ranges and values there between including 3.0 to 3.2 bar, 3.2 to 3.4 bar, 3.4 to 3.6 bar, 3.6 to 3.8 bar, 3.8 to 4.0 bar, 4.0 to 4.2 bar, 4.2 to 4.4 bar, 4.4 to 4.6 bar, 4.6 to 4.8 bar, and 4.8 to 5.0 bar. In embodiments of the invention, first intermediate stream 31 comprises 60 to 80 wt. % butadiene, and 20 to 40 wt. % combined butenes and butane. First intermediate stream 31 may further comprise 0.1 to 0.5 wt. % C₅+ hydrocarbons.

According to embodiments of the invention, as shown in block 205, separating at block 203 includes separating first intermediate stream 31 in second extractive distillation unit 106 to produce fifth stream 25 comprising primarily butenes and butane and second intermediate stream 32 comprising butadiene, butenes, butane, C₅+ hydrocarbons, a second solvent, or combinations thereof. In embodiments of the invention, second extractive distillation unit 106 uses the second solvent to separate first intermediate stream 31. The second solvent can include N-Methyl-2-pyrrolidone, 5 to 11% water, or any combination thereof. In embodiments of the invention, second extractive distillation unit 106 is operated at a temperature in a range of 80 to 100° C. and all ranges and values there between including ranges of 80 to 82° C., 82 to 84° C., 84 to 86° C., 86 to 88° C., 88 to 90° C., 90 to 92° C., 92 to 94° C., 94 to 96° C., 96 to 98° C., and 98 to 100° C. Second extractive distillation unit 106 may be operated at a pressure of 3 to 5 bar and all ranges and values there between including ranges of 3.0 to 3.2 bar, 3.2 to 3.4 bar, 3.4 to 3.6 bar, 3.6 to 3.8 bar, 3.8 to 4.0 bar, 4.0 to 4.2 bar, 4.2 to 4.4 bar, 4.4 to 4.6 bar, 4.6 to 4.8 bar, and 4.8 to 5.0 bar. Second extractive distillation unit 106 may be operated at a feed (first intermediate stream 31) to solvent (the second solvent) flow rate ratio of 0.1 to 0.2 and all ranges and values there between including ranges of 0.1 to 0.11, 0.11 to 0.12, 0.12 to 0.13, 0.13 to 0.14, 0.14 to 0.15, 0.15 to 0.16, 0.16 to 0.17, 0.17 to 0.18, 0.18 to 0.19, and 0.19 to 0.2. In embodiments of the invention, second intermediate stream 32 comprises 80 to 95 wt. % butadiene, 10 to 20 wt. % butenes and butane combined, and 0.1 to 0.5 wt. % C₅+ hydrocarbons.

According to embodiments of the invention, as shown in block 206, separating at block 203 includes separating the butenes and/or butane from second intermediate stream 32 in rectifier zone 107 to produce third intermediate stream 33 comprising butadiene, the second solvent, and C₅+ hydrocarbons. In embodiments of the invention, third intermediate stream 33 may further comprise less than 10 to 20 wt. % combined butenes and butane. In embodiments of the invention, rectifier zone 107 is operated at a temperature in a range of 80 to 100° C. and all ranges and values there between. In embodiments of the invention, separating at block 206 further produces first loop stream 42 comprising primarily C₄ hydrocarbons and the second solvent, collectively, flowing from rectifier zone 107 to second degasser 110. According to embodiments of the invention, separating at block 206 further produces second flow back stream 43 comprising non-butadiene C₄ hydrocarbons and the second solvent, flowing from rectifier zone to second extractive distillation unit 106. Rectifier zone 107 may be operated at a pressure of 3 to 5 bar and all ranges and values there between including ranges of 3.0 to 3.2 bar, 3.2 to 3.4 bar, 3.4 to 3.6 bar, 3.6 to 3.8 bar, 3.8 to 4.0 bar, 4.0 to 4.2 bar, 4.2 to 4.4 bar, 4.4 to 4.6 bar, 4.6 to 4.8 bar, and 4.8 to 5.0 bar.

According to embodiments of the invention, as shown in block 207, separating at block 203 includes separating third intermediate stream 33 in after-washer zone 108 to produce (1) fourth intermediate stream 34 comprising butadiene and C₅+ hydrocarbons and (2) first flow back stream 41 comprising C₄ hydrocarbons and the second solvent. First flow back stream 41 may be flowed back to rectifier zone 107. In embodiments of the invention, fourth intermediate stream 34 comprises 95 to 98 wt. % butadiene and all ranges and values there between including ranges of 95 to 95.5 wt. %, 95.5 to 96 wt. %, 96 to 96.5 wt. %, 96.5 to 97 wt. %, 97 to 97.5 wt. %, and 97.5 to 98 wt. %. After-washer 108 zone may be operated at a temperature in a range of 80 to 100° C. and all ranges and values there between including ranges of 80 to 82° C., 82 to 84° C., 84 to 86° C., 86 to 88° C., 88 to 90° C., 90 to 92° C., 92 to 94° C., 94 to 96° C., 96 to 98° C., and 98 to 100° C. After-washer zone 108 may be operated at a pressure in a range of 3 to 5 bar and all ranges and values there between including ranges of 3.0 to 3.2 bar, 3.2 to 3.4 bar, 3.4 to 3.6 bar, 3.6 to 3.8 bar, 3.8 to 4.0 bar, 4.0 to 4.2 bar, 4.2 to 4.4 bar, 4.4 to 4.6 bar, 4.6 to 4.8 bar, and 4.8 to 5.0 bar.

According to embodiments of the invention, as shown in block 208, separating at block 203 includes separating fourth intermediate stream 34 in second distillation unit 109 to produce third stream 23 comprising primarily butadiene and sixth stream 26 comprising primarily C₅+ hydrocarbons. In embodiments of the invention, third stream 23 comprises 99 to 99.6 wt. % butadiene and all ranges and values there between including 99 to 99.1 wt. %, 99.1 to 99.2 wt. %, 99.2 to 99.3 wt. %, 99.3 to 99.4 wt. %, 99.4 to 99.5 wt. %, and 99.5 to 99.6 wt. %. Second distillation unit 109 may be operated at a temperature of 60 to 80° C. and all ranges and values there between including ranges of 60 to 62° C., 62 to 64° C., 64 to 66° C., 66 to 68° C., 68 to 70° C., 70 to 72° C., 72 to 74° C., 74 to 76° C., 76 to 78° C., and 78 to 80° C. Second distillation unit 109 may be operated at a pressure in a range of 3 to 5 bar and all ranges and values there between including ranges of 3.0 to 3.2 bar, 3.2 to 3.4 bar, 3.4 to 3.6 bar, 3.6 to 3.8 bar, 3.8 to 4.0 bar, 4.0 to 4.2 bar, 4.2 to 4.4 bar, 4.4 to 4.6 bar, 4.6 to 4.8 bar, and 4.8 to 5.0 bar.

According to embodiments of the invention, method 200 further comprises separating first stream 21 comprising primarily soybean oil and the unreacted n-butane in first degasser 104 to produce (a) seventh stream 27 comprising primarily unreacted n-butane and (b) eighth stream 28 comprising primarily soybean oil. First degasser 104 may be operated at a temperature in a range of 50 to 80° C. and a pressure in a range of 0.5 to 2 bar. Method 200 may further comprise recycling seventh stream 27 to dehydrogenation zone of reaction unit 101 and/or recycling eighth stream 28 to first extractive distillation unit 103 as the solvent.

According to embodiments of the invention, method 200 further comprises separating from first loop stream 42 from rectifier zone in second degasser 110 to produce recycle solvent stream 15 comprising primarily the second solvent, after-washer solvent stream 14 comprising the second solvent, and recycle C₄ stream 16 comprising C₄ hydrocarbons and the second solvent. In embodiments of the invention, second degasser 110 may be operated at a temperature in a range of 100 to 160° C. and a pressure in a range of 0.5 to 2 bar.

In embodiments of the invention, method 200 further comprises recycling recycle solvent stream 15 to second extractive distillation unit 106. Method 200 may further still comprise recycling after-washer solvent stream 14 to after-washer zone 108. Method 200 may further still comprise recycling recycle C₄ stream 16 to rectifier zone 107. Method 200 may further comprise separating recycle C₄ stream 16 in rectifier zone 107 such that at least some butadiene from recycle C₄ stream 16 is flowed in third intermediate stream 33 and/or at least some non-butadiene C₄ hydrocarbons from recycle C₄ stream 16 is flowed in second flow back stream 43. In embodiments of the invention, recycle solvent stream 15 is cooled before it is recycled to second extractive distillation unit 106 and/or after-washer zone 108. In embodiments of the invention, at least a portion of recycle solvent stream 15 is fed to a solvent regeneration zone to regenerate the second solvent.

Although embodiments of the present invention have been described with reference to blocks of FIG. 2, it should be appreciated that operation of the present invention is not limited to the particular blocks and/or the particular order of the blocks illustrated in FIG. 2. Accordingly, embodiments of the invention may provide functionality as described herein using various blocks in a sequence different than that of FIG. 2.

In the context of the present invention, at the least the following 20 embodiments are described 1. Embodiment 1 is a method which includes providing a mixture containing butadiene and n-butane. The method further includes contacting the mixture with soybean oil, in a first extractive distillation unit, under conditions such that the n-butane dissolves in the soybean oil at a higher rate than the butadiene to form (1) a first stream containing the soybean oil and at least some of the n-butane of the mixture and (2) a second stream containing primarily butadiene. Embodiment 2 is the method of embodiment 1, wherein the providing step includes dehydrogenating n-butane in the presence of a catalyst capable of catalyzing double-dehydrogenation of butane under reaction conditions sufficient to produce the mixture containing butadiene and unreacted n-butane. Embodiment 3 is the method of embodiment 2, wherein the catalyst contains a Column 13 or Column 14 metal or oxide thereof and a noble metal deposited on an iron alkaline earth metal-silicon oxide support. Embodiment 4 is the method of any of embodiments 1 to 3, wherein the dehydrogenating is carried out at a temperature of 450 to 600° C., a pressure of 0.1 to 1 MPa, and a weighted hourly space velocity of 1000 to 3000 hr⁻¹. Embodiment 5 is the method of any of embodiments 1 to 4, wherein the conditions of the contacting step include a temperature of 10 to 50° C. and a pressure of 3 to 5 bar. Embodiment 6 is the method of any of embodiments 1 to 5, wherein the mixture further contains C₁ to C₃ hydrocarbons and C₅+ hydrocarbons. Embodiment 7 is the method of any of embodiments 1 to 6, further including separating the second stream into a third stream containing primarily butadiene, a fourth stream containing C₁ to C₃ hydrocarbons, a fifth stream containing primarily butane and butenes, and a sixth stream containing hydrocarbons having a higher boiling point than butadiene, including C₅+ hydrocarbons. Embodiment 8 is the method of embodiment 7, wherein the separating of the second stream includes separating the second stream to produce the fourth stream containing C₁ to C₃ hydrocarbons and a first intermediate stream containing primarily butadiene, butane, and butenes, collectively. The method further includes separating the first intermediate stream in a second extractive distillation unit to produce the fifth stream containing primarily butenes and butane and a second intermediate stream containing butadiene, butenes, butane, C₅+ hydrocarbons, and a second solvent. The method also includes separating the butenes from the second intermediate stream in a rectifier zone to produce a third intermediate stream containing butadiene, the second solvent, and C₅+ hydrocarbons. In addition, the method includes separating the third intermediate stream in an after-washer zone to produce a fourth intermediate stream containing butadiene and a solvent rich stream containing primarily the second solvent. The method further includes separating the fourth intermediate stream to produce the third stream containing primarily butadiene and the sixth stream containing primarily C₅+ hydrocarbons. Embodiment 9 is the method of embodiment 8, wherein the separating of the second stream to produce the fourth stream and the first intermediate stream is carried out in a first distillation column. Embodiment 10 is the method of embodiment 9, wherein the first distillation column is operated at a temperature of 75 to 85° C. and a pressure of 3 to 5 bar. Embodiment 11 is the method of any of embodiments 8 to 10, wherein the second solvent in the second extractive distillation unit contains N-Methyl-2-pyrrolidone. Embodiment 12 is the method of any of embodiments 8 to 11, wherein the rectifier zone and the after-washer zone are integrated in a dividing wall column. Embodiment 13 is the method of any of embodiments 8 to 12, wherein the solvent rich stream further contains less than 10 to 20 wt. % C₄ hydrocarbons. Embodiment 14 is the method of embodiment 13, further including removing C₄ hydrocarbons from the solvent rich stream in a second degasser to produce a recycle solvent stream containing primarily the second solvent. The method further includes recycling the recycle solvent stream to the after-washer zone and/or the second extractive distillation unit. Embodiment 15 is the method of embodiment 14, wherein the recycle solvent stream from the second degasser is cooled before it is recycled to the after-washer zone and/or the second extractive distillation unit. Embodiment 16 is the method of either of embodiments 14 or 15, wherein at least a portion of the recycle solvent stream is fed to a solvent regeneration zone. Embodiment 17 is the method of any of embodiments 8 to 16, wherein the third stream contains 99 to 99.9 wt. % butadiene. Embodiment 18 is the method of any of embodiments 8 to 17, wherein the fourth intermediate stream is separated in a distillation column. Embodiment 19 is the method of any of embodiments 1 to 18, further including separating the first stream in a first degasser to produce a seventh stream containing primarily n-butane and an eighth stream containing primarily soybean oil. Embodiment 20 is the method of embodiment 19, further including recycling the seventh stream to a dehydrogenation zone that carries out the dehydrogenating of n-butane, and recycling the eighth stream to the first extractive distillation unit.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method comprising:
providing a mixture comprising butadiene and n-butane; and contacting the mixture with soybean oil, in a first extractive distillation unit, under conditions such that the n-butane dissolves in the soybean oil at a higher rate than the butadiene to form (1) a first stream comprising the soybean oil and at least some of the n-butane of the mixture and (2) a second stream comprising primarily butadiene.

2. The method of claim 1, wherein the providing step comprises dehydrogenating n-butane in the presence of a catalyst capable of catalyzing double-dehydrogenation of butane under reaction conditions sufficient to produce the mixture comprising butadiene and unreacted n-butane.

3. The method of claim 2, wherein the catalyst comprises a Column 13 or Column 14 metal or oxide thereof and a noble metal deposited on an iron alkaline earth metal-silicon oxide support.

4. The method of claim 1, wherein the dehydrogenating is carried out at a temperature of 450 to 600° C., a pressure of 0.1 to 1 MPa, and a weighted hourly space velocity of 1000 to 3000 hr⁻¹.

5. The method of claim 1, wherein the conditions of the contacting step comprise a temperature of 10 to 50° C. and a pressure of 3 to 5 bar.

6. The method of claim 1, wherein the mixture further comprises C₁ to C₃ hydrocarbons, and C₅+ hydrocarbons.

7. The method of claim 1, further comprising separating the second stream into a third stream comprising primarily butadiene, a fourth stream comprising C₁ to C₃ hydrocarbons, a fifth stream comprising primarily butane and butenes, and a sixth stream comprising hydrocarbons having a higher boiling point than butadiene including C₅+ hydrocarbons.

8. The method of claim 7, wherein the separating of the second stream comprises:
separating the second stream to produce the fourth stream comprising C₁ to C₃ hydrocarbons and a first intermediate stream comprising primarily butadiene, butane, and butenes, collectively;
separating the first intermediate stream in a second extractive distillation unit to produce the fifth stream comprising primarily butenes and butane and a second intermediate stream comprising butadiene, butenes, butane, C₅+ hydrocarbons, and a second solvent;
separating the butenes from the second intermediate stream in a rectifier zone to produce a third intermediate stream comprising butadiene, the second solvent, and C₅+ hydrocarbons;
separating the third intermediate stream in an after-washer zone to produce a fourth intermediate stream comprising butadiene and a solvent rich stream comprising primarily the second solvent; and
separating the fourth intermediate stream to produce the third stream comprising primarily butadiene and the sixth stream comprising primarily C₅+ hydrocarbons.

9. The method of claim 8, wherein the separating of the second stream to produce the fourth stream and the first intermediate stream is carried out in a first distillation column.

10. The method of claim 9, wherein the first distillation column is operated at a temperature of 75 to 85° C. and a pressure of 3 to 5 bar.

11. The method of 8, wherein the second solvent in the second extractive distillation unit comprises N-Methyl-2-pyrrolidone.

12. The method of 8, wherein the rectifier zone and the after-washer zone are integrated in a dividing wall column.

13. The method of 8, wherein the solvent rich stream further comprises less than 10 to 20 wt. % C₄ hydrocarbons.

14. The method of claim 13, further comprising:
removing C₄ hydrocarbons from the solvent rich stream in a second degasser to produce a recycle solvent stream comprising primarily the second solvent; and
recycling the recycle solvent stream to the after-washer zone and/or the second extractive distillation unit.

15. The method of claim 14, wherein the recycle solvent stream from the second degasser is cooled before it is recycled to the after-washer zone and/or the second extractive distillation unit.

16. The method of claim 14, wherein at least a portion of the recycle solvent stream is fed to a solvent regeneration zone.

17. The method of claim 8, wherein the third stream comprises 99 to 99.9 wt. % butadiene.

18. The method of claim 8, wherein the fourth intermediate stream is separated in a distillation column.

19. The method of claim 8, further comprising separating the first stream in a first degasser to produce a seventh stream comprising primarily n-butane and an eighth stream comprising primarily soybean oil.

20. The method of claim 19, further comprising:
recycling the seventh stream to a dehydrogenation zone that carries out the dehydrogenating of n-butane; and
recycling the eighth stream to the first extractive distillation unit.
